(19) United States
(12) Patent Application Publication
(10) Pub. No.: US 2006/0002989 A1
(43) Pub. Date: Jan. 5, 2006

(54) Formulations of sumatriptan for absorption across biological membranes, and methods of making and using the same

(76) Inventors: Salah U. Ahmed, New City, NY (US); Lianli Li, Pomona, NY (US); Venkatesh Naini, Parsippany, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/149,444

(22) Filed: Jun. 10, 2005

Related U.S. Application Data
(60) Provisional application No. 60/578,286, filed on Jun. 10, 2004.

(51) Int. Cl. A61K 31/716; A61L 15/16

(52) U.S. Cl. 424/448; 514/57

(57) ABSTRACT

The present invention is directed to pharmaceutical compositions comprising sumatriptan succinate and sodium caprate for increased absorption of sumatriptan succinate across biological membranes. The invention is also directed to methods of making the pharmaceutical compositions and uses thereof.

This application claims the benefit of the filing date of U.S. Appl. 60/578,286, filed Jun. 10, 2004, which is incorporated herein in its entirety.

### BACKGROUND OF THE INVENTION

1. Field of the Invention

The present invention is directed to pharmaceutical compositions comprising sumatriptan succinate and sodium caprate for increased absorption of sumatriptan succinate across biological membranes. The invention is also directed to methods of making the pharmaceutical compositions and uses thereof.

2. Background Art

Sumatriptan is a selective 5-hydroxytryptamine ID (5-HT_{1D}) receptor agonist useful for treatment of migraine. Sumatriptan is also known as 3-[2-(dimethylamino)ethyl]-N-methyl-indole-5-methanesulphonamide.

Pharmaceutical preparations containing sumatriptan or salts of sumatriptan are described in U.S. Pat. Nos. 4,816,470; 4,994,483; 5,037,845; 5,270,333; 5,288,498; 5,307,953; 5,393,773; 5,447,729; 5,554,639; 5,705,520; 5,863,559; 6,020,001; 6,255,502; 6,294,192; and 6,368,627; U.S. Patent Appl. Pub. Nos. 2003/0013753; 2003/0185761; and 2003/0190286; WO 98/02186; WO 01/39836; and DE 4314976.

An injectable form of sumatriptan succinate, Formula I, was approved by the U.S. Food and Drug Administration (FDA) for acute treatment of migraine attacks, with or without aura, and the acute treatment of cluster headache episodes:

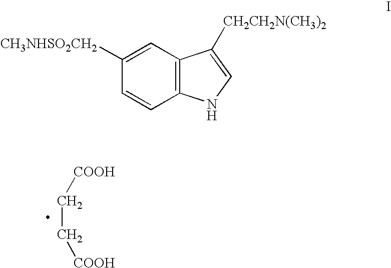

An oral tablet form of sumatriptan succinate and a nasal spray form of sumatriptan base were also approved by the FDA for acute treatment of migraine attacks, with or without aura.

Although subcutaneous injection of sumatriptan succinate provides rapid migraine control, it is an invasive method of administration and is disliked and poorly tolerated by many patients. Administration of an oral tablet of sumatriptan succinate is sometimes unsuitable for patients because it can cause severe migraine-related vomiting. The intranasal spray currently available does not significantly improve the bioavailability of sumatriptan. The bioavailability of sumatriptan using the nasal spray is 17% whereas it is 15% for the oral tablet. Following mucociliar clearance, sumatriptan administered by the intranasal spray is primarily absorbed by the oral route after 10-15 min of residence time on nasal mucosa. Further, nasal absorption is associated with high variability for patients suffering from cold or allergy. There remains a need for pharmaceutical compositions of sumatriptan with ease of administration and improved bioavailability.

### BRIEF SUMMARY OF THE INVENTION

The present invention is directed to a method of making a pharmaceutical composition for rapid transmucosal delivery comprising sumatriptan succinate and sodium caprate, the method comprising mixing sumatriptan succinate and sodium caprate to form a mixture, wherein a molar ratio (M) of a molar concentration of sodium caprate to a molar concentration of sumatriptan succinate is about 0.1 or greater, wherein absorption of sumatriptan succinate across a biological membrane (Fₛ) is equal to F₀+κln(M), wherein F₀ is a steady state flux value of the absorption when the molar ratio of a molar concentration of sodium caprate to a molar concentration of sumatriptan succinate is 1, and wherein κ is an enhancement factor. In some embodiments, the method further comprises compressing the mixture into a pharmaceutical composition, wherein the mixture can be a dry mixture, a wet granulate, a gel, a paste, a solution or combinations thereof.

The invention is also directed to a method of making a pharmaceutical composition for rapid transmucosal delivery comprising sumatriptan succinate and sodium caprate, the method comprising dispersing sumatriptan succinate and sodium caprate in water or a solvent to prepare a mixture, and casting the mixture to form a pharmaceutical composition, wherein a molar ratio (M) of a molar concentration of sodium caprate to a molar concentration of sumatriptan succinate is about 0.1 or greater, wherein absorption of sumatriptan succinate across a biological membrane (Fₛ) is equal to F₀+κln(M), wherein F₀ is a steady state flux value of the absorption when the molar ratio of a molar concentration of sodium caprate to a molar concentration of sumatriptan succinate is 1, and wherein the κ is an enhancement factor. In some embodiments, the mixture is spray dried to form a second mixture. In some embodiments, the method further comprises compressing the second mixture into a pharmaceutical composition.

The invention is also directed to a method of treating migraine, the method comprising administering a pharmaceutical composition comprising sumatriptan succinate and sodium caprate, wherein a molar ratio (M) of a molar concentration of sodium caprate to a molar concentration of sumatriptan succinate is about 0.1 or greater, wherein the absorption of sumatriptan succinate across a biological membrane (Fₛ) is equal to F₀+κln(M), wherein F₀ is a steady state flux value of the absorption when the molar ratio of a molar concentration of sodium caprate to a molar concentration of sumatriptan succinate is 1, and wherein κ is an enhancement factor, to a person in need of the treatment. In some embodiments, sodium caprate is the absorption enhancer.

The invention is also directed to a method of treating cluster headache episodes, the method comprising administering a pharmaceutical composition comprising sumatriptan succinate and sodium captrate, wherein a molar ratio (M) of a molar concentration of sodium caprate to a molar concentration of sumatriptan succinate is about 0.1 or greater, wherein the absorption of sumatriptan succinate across a biological membrane (Fₛ) is equal to F₀+κln(M), wherein F₀ is a steady state flux value of the absorption when the molar ratio of a molar concentration of sodium caprate to a molar concentration of sumatriptan succinate is 1, and wherein κ is an enhancement factor, to a person in need of the treatment

The present invention is also directed to a pharmaceutical composition for rapid transmucosal delivery comprising sumatriptan succinate and sodium caprate, wherein the molar concentration of the sodium caprate is about 1 μM to about 250 mM.

The present invention is also directed to a pharmaceutical composition for rapid transmucosal delivery comprising sumatriptan succinate and sodium caprate, wherein the amount of sodium caprate per dosage unit is about 1 μmol to about 250 mmol.

### BRIEF DESCRIPTION OF THE FIGURES

FIG. 1 provides a process flow chart for a method of manufacturing the pharmaceutical compositions of the invention by a dry mixing process.

FIG. 2 provides a process flow chart for a method of manufacturing the pharmaceutical compositions of the invention by a wet granulation process.

FIG. 3 is a graph that shows the relationship between the steady state flux of absorption (Fₛ) of sumatriptan succinate across a buccal membrane and the molar ratio (M) of the molar concentration of sodium caprate to the molar concentration of sumatriptan succinate. The graph shows ln(M) on the x-axis and Fₛ (ng/cm²/min) on the y-axis.

FIG. 4A provides a chromatogram from a high performance liquid chromatography (HPLC) analysis of a standard solution of sumatriptan succinate alone. The x-axis shows retention time (mins) and the y-axis shows absorbance (in absorbance units (AU)) at 288 nm.

FIG. 4B provides a chromatogram from an HPLC analysis of a sample taken from the receptor-side of a side-by-side diffusion experiment. The x-axis shows retention time (mins) and the y-axis shows absorbance (AU) at 288 nm.

FIG. 4C provides absorbance data for the major peak seen in FIG. 4A. The x-axis shows wavelength (nm) and the y-axis shows AU.

FIG. 4D provides absorbance data for the major peak seen in FIG. 4B. The x-axis shows wavelength (nm) and the y-axis shows AU.

FIG. 5 provides a graph showing the relationship between the latent heat of fusion, ΔH_{f}, and the weight percent of sumatriptan succinate in samples containing varying amounts of sumatriptan succinate. The x-axis shows the weight % (wt %) of sumatriptan succinate and the y-axis shows ΔH_{f} in joules/gram (J/g) from the peak observed at about 170° C. during differential scanning calorimetry (DSC).

FIG. 6A provides a chromatogram from an HPLC analysis of a standard solution of sumatriptan succinate alone. The x-axis shows retention time (mins) and the y-axis shows absorbance (AU) at 288 nm.

FIG. 6B provides a chromatogram from an HPLC analysis of a dry mixture of sumatriptan succinate and sodium caprate. The x-axis shows retention time (mins) and the y-axis shows absorbance (AU) at 288 nm.

FIG. 6C provides absorbance data for the major peak seen in FIG. 6A. The x-axis shows wavelength (nm) and the y-axis shows AU.

FIG. 6D provides absorbance data for the major peak seen in FIG. 6B. The x-axis shows wavelength (nm) and the y-axis shows AU.

FIG. 7A provides a chromatogram from an HPLC analysis of a standard solution of sumatriptan succinate alone. The x-axis shows retention time (mins) and the y-axis shows absorbance (AU) at 288 nm.

FIG. 7B provides a chromatogram from an HPLC analysis of a wet mixture of sumatriptan succinate and sodium caprate. The x-axis shows retention time (mins) and the y-axis shows absorbance (AU) at 288 nm.

FIG. 7C provides absorbance data for the major peak seen in FIG. 7A. The x-axis shows wavelength (nm) and the y-axis shows AU.

FIG. 7D provides absorbance data for the major peak seen in FIG. 7B. The x-axis shows wavelength (nm) and the y-axis shows AU.

FIG. 8 provides a plasma concentration versus time curve for a sumatriptan study performed in dogs.

### DETAILED DESCRIPTION OF THE INVENTION

The invention is directed to a pharmaceutical composition for rapid transmucosal delivery comprising a drug (e.g., sumatriptan succinate) and an absorption enhancer (e.g., sodium caprate) wherein a molar ratio (M) of a molar concentration of the absorption enhancer to a molar concentration of the drug is about 0.1 or greater, wherein absorption of the drug across a biological membrane (Fₛ) is equal to F₀+κln(M), wherein F₀ is a steady state flux value of the absorption when the molar ratio of a molar concentration of the absorption enhancer to a molar concentration of the drug is 1, and wherein κ is an enhancement factor.

The drug in the pharmaceutical compositions include but are not limited to inorganic and organic salts of sumatriptan such as hydrochloride, hydrobromide, sulphate, nitrate, phosphate, formate, mesylate, citrate, benzoate, fumarate, maleate, tartrate, hemisuccinate, methanesulphonate, succinate, and combinations thereof. In some embodiments the drug is sumatriptan succinate.

Absorption of a drug involves passage of the drug across biological membranes whereby a cell, tissue or organ takes up the drug. Absorption is also referred to as the rate and extent to which a drug leaves its site of administration. The physicochemical properties of the molecules in the pharmaceutical composition as well as that of the membranes affect the absorption of drugs across membranes.

Biological membranes are sheets of tissue that include but are not limited to membranes that provide a pliable surface lining for protecting or partitioning organs and structures in the body. As used herein, a biological membrane is an epithelial membrane. Epithelial membranes include but are not limited to coverings or linings of the outer layer of skin and some internal organs, e.g., digestive, respiratory, reproductive and urinary systems. Epithelial membranes include the lining of body cavities. Epithelial membranes include but are not limited to oral, buccal, sublingual, gingival, palatal, nasal, nasopharynxal, oropharynxal, conjunctival, transdermal, vaginal and gastrointestinal membranes. In some embodiments, the structure can be a cellular structure.

In some embodiments, the biological membrane is a buccal mucosal membrane. In some embodiments, the invention is directed to a pharmaceutical composition comprising sumatriptan succinate and sodium caprate in dosage forms suitable for increased absorption across a buccal mucosal membrane. The advantages of buccal delivery are that it bypasses the first-pass effect associated with peroral delivery of sumatriptan, provides for ease of administration, and provides for the likelihood of rapid-onset of anti-migraine effect.

In some embodiments, the invention is directed to pharmaceutical compositions for the rapid transmucosal delivery of a drug, e.g., sumatriptan. Rapid transmucosal delivery means that the drug is delivered transmucosally with a rate of absorption which exceeds that of the sumatriptan oral tablet (IMITREX).

Absorption enhancers are agents that increase drug absorption across biological membranes. Absorption enhancers for use in pharmaceutical compositions of the present invention include but are not limited to sodium caprate, sodium caprylate, sodium laurate, sodium lauryl sulfate and combinations thereof.

In some embodiments, the absorption enhancer is sodium caprate. In some embodiments, the molar concentration of sodium caprate can be about 1 μM to about 250 mM. In some embodiments, the molar concentration of sodium caprate can be about 1 mM to about 200 mM. In some embodiments, the molar concentration of sodium caprate can be about 1 mM to about 150 mM. In some embodiments, the molar concentration of sodium caprate can be about 10 mM to about 250 mM. In some embodiments, the molar concentration of sodium caprate can be about 10 mM to about 200 mM. In some embodiments, the molar concentration of sodium caprate can be about 10 mM to about 100 mM. In some embodiments, the molar concentration of sodium caprate can be about 10 mM to about 80 mM.

In some embodiments, the amount of sodium caprate per dosage unit is about 1 μmol to about 250 mmol. In some embodiments, the amount of sodium caprate per dosage unit is about 1 mmol to about 200 mmol. In some embodiments, the amount of sodium caprate per dosage unit is about 1 mmol to about 150 mmol. In some embodiments, the amount of sodium caprate per dosage unit is about 10 mmol to about 250 mmol. In some embodiments, the amount of sodium caprate per dosage unit is about 10 mmol to about 200 mmol. In some embodiments, the amount of sodium caprate per dosage unit is about 10 mmol to about 100 mmol.

The molar ratio (M) is the molar concentration of the absorption enhancer to the molar concentration of the drug, e.g., sumatriptan succinate. In some embodiments, the value of M is about 0.1 or greater, or about 0.5 or greater, or about 1.0 or greater. In some embodiments, the value of M is about 0.1 to about 15. In some embodiments, the value of M is about 0.5 to about 10. In some embodiments, the value of M is about 0.1 to about 15. In some embodiments, the value of M is about 0.5 to about 10. In some embodiments, the value of M is about 0.8 to about 7. In some embodiments, the value of M is about 1.2 to about 7. In some embodiments, the value of M is about 1.5 to about 7.

Fₛ is a steady state flux value of the absorption of a drug, e.g., sumatriptan succinate, across a biological membrane and is equal to F₀+κln(M). The value of Fₛ can be determined experimentally for the pharmaceutical compositions of the present invention, e.g., by performing side-by-side diffusion experiments over a varying range of molar ratios of the molar concentration of the absorption enhancer to the molar concentration of the drug (e.g., sumatriptan succinate) while maintaining substantially constant the physicochemical characteristics of the biological membrane. In a side-by-side diffusion experiment, there exists a reservoir on each side of the test biological membrane. One reservoir, referred to as the donor-side, contains the drug with or without the absorption enhancer, whereas the other side, referred to as the receptor-side, has only buffer. The diffusion of drug, e.g., sumatriptan succinate, from the donor-side to the receptor-side is monitored over a period of time. The steady state flux of absorption, Fₛ, is calculated by the formula, Fₛ=(dQ/dt)/A=(dC/dt)×(V/A), wherein Q is the amount of drug permeated, C is the concentration of the drug, A is the permeation area, V is the chamber volume in a side-by-side diffusion cell, and t is the time period over which the drug permeation is monitored. Fₛ can be determined from the slope of the line attained from plotting the cumulative amount of drug permeated per unit area as a function of time.

The results of such an experiment measuring the absorption of a drug, e.g., sumatriptan succinate, across a biological membrane can be expressed as Fₛ as a function of ln(M). The value of Fₛ can vary depending on, e.g., the molar ratio (M) of the pharmaceutical composition, the physicochemical properties of the biological membrane such as thickness and type of the membrane, the diffusion medium used in the experiments, the concentration of drug in the donor-side in side-by-side diffusion experiments, the speed at which the solutions are stirred in a side-by-side diffusion experiment, the volume of sample removed from the receptor-side for sampling, and the frequency with which the samples are removed from the receptor-side for sampling.

F₀ is a steady state flux value of absorption of drug (e.g., sumatriptan succinate) across a biological membrane when the value of the molar ratio of a molar concentration of the absorption enhancer (e.g., sodium caprate) to the molar concentration of drug is 1. F₀ is constant for a given experimental condition and is the intercept of Fₛ (y-axis) versus ln(M) (x-axis).

The value of F₀ can be determined experimentally, e.g., by performing side-by-side diffusion experiments measuring the permeability of the drug across a biological membrane for the pharmaceutical composition of the invention. However, the specific value of F₀ can vary depending on changes in the experimental conditions, e.g., the physicochemical properties of the biological membrane, the diffusion medium used in the experiments, the concentration of drug in the donor-side in side-by-side diffusion experiments, the speed at which the solutions are stirred in a side-by-side diffusion experiment, the volume of sample removed from the receptor-side for sampling, and the frequency with which the samples are removed from the receptor-side for sampling. In some embodiments, when, e.g., sodium caprate is the absorption enhancer, the value of F₀ can be about 100 ng/cm²/min to about 1000 ng/cm²/min, about 150 ng/cm²/min to about 950 ng/cm²/min, about 300 ng/cm²/min to about 550 ng/cm²/min, or about 420 ng/cm²/min.

The enhancement factor κ is constant for a given experimental condition and is the slope of Fₛ (y-axis) versus ln(M) (x-axis). The value of κ can be determined experimentally, e.g., by performing side-by-side diffusion experiments for the pharmaceutical compositions of the invention. However, the value of κ can vary depending on changes in the experimental conditions, e.g., the physicochemical properties of the biological membrane, the diffusion medium used in the experiments, the concentration of drug in the donor-side in side-by-side diffusion experiments, the speed at which the solutions are stirred in a side-by-side diffusion experiment, the volume of sample removed from the receptor-side for sampling, and the frequency with which the samples are removed from the receptor-side for sampling. In some embodiments, when, e.g., sodium caprate is the absorption enhancer, the value of κ can be about 1000 ng/cm²/min to about 2000 ng/cm²/min, about 1200 ng/cm²/min to about 1500 ng/cm²/min, or about 1300 ng/cm²/min.

The absorption data obtained for a range of molar ratios can be subjected to regression analysis. Regression analysis is a group of statistical methods to examine the degree of association between one variable (or set of variables) and another variable (or set of variables). Regression analysis methods are generally described in Remington: The Science and Practice of Pharmacy, Lippincott Williams & Wilkins, 21^{st} ed. (2004). Regression analysis of the absorption data provides a range of values for a correlation coefficient (r). The correlation coefficient provides a measure of the relationship between the two variables. For the pharmaceutical compositions of the invention, determining the correlation coefficient provides a measure of the relationship between the absorption of drug (e.g., sumatriptan succinate) across a biological membrane and the natural logarithmic value of the molar ratio of the molar concentration of an absorption enhancer to the molar concentration of sumatriptan succinate (ln(M)). In some embodiments, when, e.g., sodium caprate is the absorption enhancer, regression analysis provides a correlation coefficient (r) of about 0.9 to about 1, or about 0.95 to about 1.

The pharmaceutical compositions of the invention can be formulated with one or more carriers or excipients, such as but not limited to hydroxypropyl cellulose, hydroxyethyl cellulose, hydroxypropylmethyl cellulose, sodium carboxymethyl cellulose, methyl cellulose, polyvinylpyrrolidone, polyethylene glycol, vegetable oil, polyols, lactose and combinations thereof. As one of skill in the are can readily determine, many carriers, e.g., polymers, can be used in the present invention depending on the molecular weight of the polymer, the viscosity of the polymer, and the amount of the polymer in the pharmaceutical composition.

The pharmaceutical compositions of the invention can include one or more mucoadhesive polymers. Mucoadhesive polymers have physicochemical properties suitable for adhering to biological membranes. Mucoadhesive polymers are natural or synthetic polymers that adhere to mucosal membranes by means of hydrogen bonds, ionic interactions, physical entanglements, and combinations thereof. In some embodiments, mucoadhesive polymers adhere to wet mucosal epithelial membranes. Mucoadhesive substances for use in the pharmaceutical compositions of the invention can include but are not limited to poly(ethylene oxide), polyvinylpyrrolidone, copovidone, carbomer, polycarbophil, hydroxypropyl cellulose, hydroxyethyl cellulose, sodium carboxymethyl cellulose, polyvinyl alcohol, and combinations thereof.

The pharmaceutical compositions of the invention can comprise one or more diluents. A diluent is any inert substance, or mixture of substances, added to increase the bulk of the pharmaceutical formulation in order to make the solid oral dosage form a practical size for administration or compression. Diluents include but are not limited to lactose, starch, polyethylene glycol, maltodextrin, dextrose, mannitol, xylitol, other polyols, and combinations thereof.

The pharmaceutical compositions of the invention can be formulated for non-parenteral administration. Exemplary non-parenteral routes include, but are not limited to, the buccal, sublingual, nasal, transdermal, oral, or other transmucosal route. The pharmaceutical compositions of this invention can also be formulated for various dosage forms that include but are not limited to a tablet, disk, patch, film, wafer, gel, paste and solution dosage forms. Suitable solution dosage forms for the present invention include, but are not limited to, a nasal spray, a nasal drop, a sublingual solution, or any other solution which can be administered transmucosally.

The invention is also directed to methods of making pharmaceutical compositions comprising a drug (e.g., sumatriptan succinate) and an absorption enhancer (e.g., sodium caprate), the method comprising mixing the drug and the absorption enhancer to form a mixture, wherein a molar ratio (M) of a molar concentration of the absorption enhancer to a molar concentration of the drug is about 0.1 or greater, wherein absorption of the drug across a biological membrane (Fₛ) is equal to F₀+κln(M), wherein F₀ is a steady state flux value of the absorption when the molar ratio of a molar concentration of the absorption enhancer to a molar concentration of the drug is 1, and wherein κ is an enhancement factor.

Methods of preparing various pharmaceutical compositions with a certain amount of active ingredients are known, or will be apparent in light of this disclosure, to those skilled in the art. Methods of preparing the pharmaceutical compositions can incorporate other suitable pharmaceutical excipients and their formulations as described in Remington: The Science and Practice of Pharmacy, Lippincott Williams & Wilkins, 21^{st} ed. (2004).

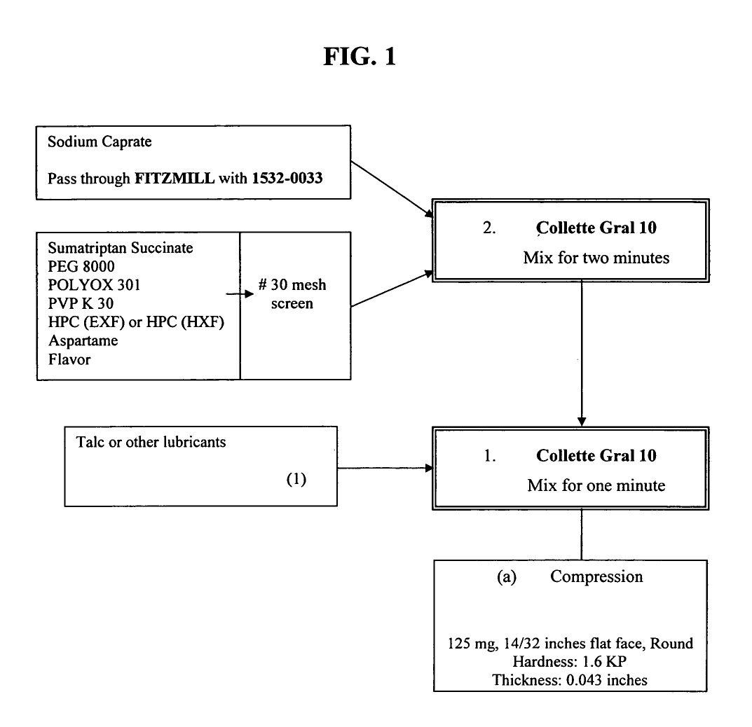

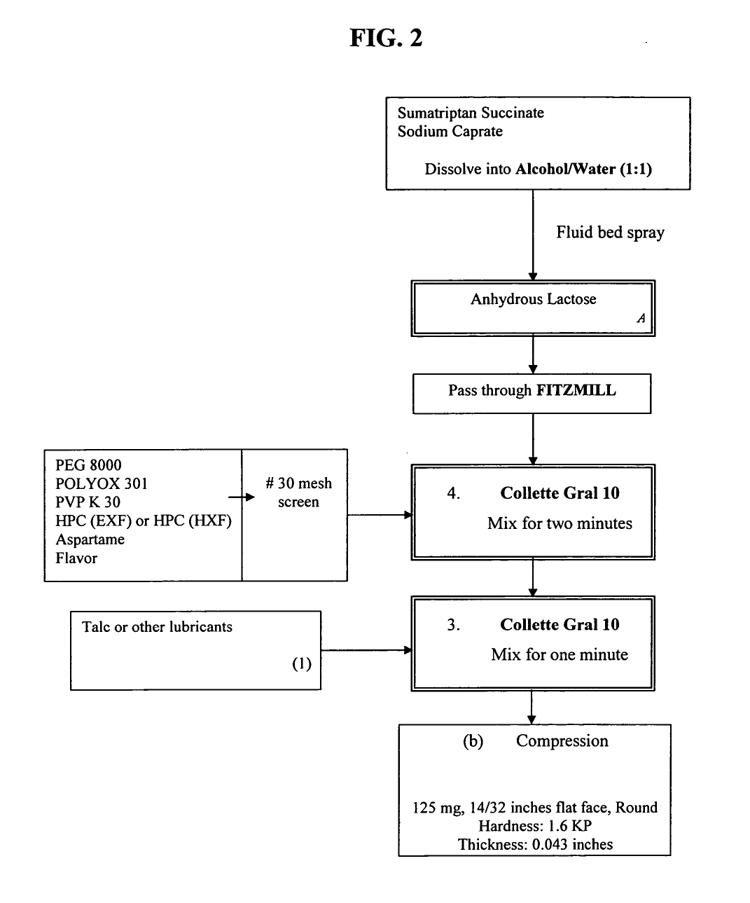

The pharmaceutical compositions of the present invention can be manufactured in a manner that is known in the art, including conventional dry or wet mixing, dissolving, or compressing processes. Pharmaceutical compositions can be obtained by combining the drug (e.g., sumatriptan succinate) and one or more absorption enhancers to form mixtures. Optionally the resulting mixture can be processed after adding suitable auxiliaries, if desired or necessary. Two exemplary methods of preparing the pharmaceutical compositions of the invention, by a dry mixing process and by a wet granulation process, are provided in FIGS. 1 and 2.

In some embodiments, the method further comprises compressing the mixture into a pharmaceutical composition, wherein the mixture is a dry mixture. In some embodiments, the method further comprises compressing the mixture into a pharmaceutical composition, wherein the mixture is a wet granulate. In some embodiments, the mixture is a gel, a paste, or a solution.

In some embodiments, the invention provides a method of making a pharmaceutical composition comprising a drug (e.g., sumatriptan succinate) and an absorption enhancer (e.g., sodium caprate), wherein the method comprises dispersing the drug and the absorption enhancer in water or a solvent to prepare a mixture, and casting the mixture to form a pharmaceutical composition, wherein a molar ratio (M) of a molar concentration of the absorption enhancer to a molar concentration of the drug is about 0.1 or greater, wherein the steady state flux of absorption of the drug across a biological membrane (Fₛ) is equal to F₀+κln(M), wherein F₀ is a steady state flux value of absorption when the molar ratio of a molar concentration of the absorption enhancer to a molar concentration of the drug is 1, and wherein κ is an enhancement factor. In some embodiments, the mixture can be spray dried to form a second mixture. In some embodiments, the method further comprises compressing the second mixture into a pharmaceutical composition.

Various solvents can be used. In some embodiments, the solvents used to prepare the mixtures include volatile or dryable solvents, such as, water, isopropanol, ethanol, methanol, acetone, ethyl acetate, and combinations thereof.

Casting of the mixture can be performed during the preparation of the pharmaceutical compositions. Casting refers to the process of spreading the mixture onto suitable devices and drying. In some embodiments, the dried components are cut into uniform pieces.

In some embodiments, the invention is directed to pharmaceutical compositions made by the methods of the invention. The enhancement of sumatriptan succinate absorption was related to the molar ratio of the molar concentration of sodium caprate to the molar concentration of sumatriptan succinate rather than to the method of preparation of the composition.

The pharmaceutical compositions of the invention can be used for the treatment of migraine attacks, with or without aura, or for the treatment of cluster headache episodes in adults as well as children. In some embodiments, the invention comprises a method of treating migraine, the method comprising administering the pharmaceutical compositions of the invention to a person in need of the treatment.

As used herein, “about” refers to plus or minus 10% of the indicated number.

All of the various embodiments or options described herein can be combined in any and all variations.

The following Examples serve only to illustrate the invention and are not to be construed in anyway to limit the invention.

### EXAMPLES

### Example 1

### Enhancement of Sumatriptan Succinate Absorption Across the Buccal Membrane

The steady state flux of absorption of sumatriptan succinate, Fₛ, across a buccal epithelial membrane ranging in thickness from about 400 nanometers to about 600 nanometers was measured. Freshly prepared buccal tissue from pigs was used to prepare the buccal epithelial membrane. A dermatometer was used to obtain the buccal epithelial membrane by separating it from the underlying connective tissue. The buccal membranes were used within 3-4 hours of removal of buccal tissue from the pigs.

The buccal epithelial membrane was mounted between side-by-side diffusion cells. The exposed area of the buccal membrane was approximately 0.64 cm². In some experiments sumatriptan base alone or sumatriptan succinate alone in Krebs-Ringer bicarbonate (KRB) solution were dosed in the donor-side. In some experiments sumatriptan base or sumatriptan succinate was mixed with absorption enhancers in KRB solution and dosed in the donor-side. The final volume of the liquid in the donor-side was 3.5 ml and the volume in the receptor-side was 3.5 ml.

The diffusion system was maintained at 37° C. throughout the experiment. At predetermined intervals over a period of 6 hours, 150 μl of the solution from the receptor-side was withdrawn for HPLC analysis. The receptor-side was refilled with the same volume (150 μl) of KRB solution.

HPLC analysis of the solution withdrawn from the receptor-side was performed using a Waters 2695 separation module HPLC system equipped with a reverse phase C₁₈ column (150 mm×3.9 mm ID, 5 μm) (Waters Corp., Milford, Mass.). The mobile phase was 88% phosphate buffer (0.05 M NH₄H₂PO₄/H₃PO₄, pH 3.3) and 12% acetonitrile. Ultraviolet (UV) analysis was performed using a Waters 2996 photodiode array detector (Waters Corp., Milford, Mass.) with the wavelength set at 228 nm to detect sumatriptan succinate. The software used for HPLC assay data analysis was Millenium 32 (Waters Corp., Milford, Mass.).

Absorption of drug, i.e., sumatriptan succinate or sumatriptan base, across the buccal membrane, Fs, was determined from the slope of the straight line (2-6 hrs) attained from the plot of the cumulative amount of sumatriptan succinate permeated as a function of time. Microsoft Excel 2000 was used to calculate the steady state flux value of the absorption of the drug across the buccal membrane. The steady state flux (Fₛ)=(dQ/dt)/A=(dC/dt)×(V/A), wherein Q is the amount of drug permeated, C is concentration of the drug, A is the permeation area, V is the chamber volume, and t is the time period over which the drug permeation is monitored.

Table 1 provides the results in which sumatriptan succinate salt (SS) or sumatriptan base was mixed with sodium caprate, sodium laurate, sodium lauryl sulfate, sodium caprylate, EDTA, sodium glycocholate, lauric acid, or with lysolecithin in KRB solution and dosed in the donor-side (3.5 ml). The receptor-side contained 3.5 ml of KRB solution.

TABLE 1
Trans-buccal absorption of sumatriptan succinate and base across
pig buccal membrane: Effect of absorption enhancers.

| | Steady-state | |
|---|---|---|
| | flux, Fₛ | Enhancement Ratio |
| Composition | (ng/cm²/min) | F_{s, SS+enhancer}/F_{s, SS alone} |
| Sumatriptan Succinate | | |
| (SS) 12 mM | | |
| Salt (SS) alone | 6.0 ± 6.6 | |
| +80 mM Na Caprate | 3068.6 ± 314.0 | 511 |
| +60 mM Na Caprate | 2379.7 ± 610.7 | 397 |
| +40 mM Na Caprate | 2083.4 ± 1071.5 | 347 |
| +20 mM Na Caprate | 1055.4 ± 506.7 | 176 |
| +10 mM Na Caprate | 210.4 ± 127.1 | 35 |
| +20 mM Na Laurate | 549.5 ± 444.3 | 92 |
| +20 mM SLS | 154.8 ± 197.9 | 26 |
| +20 mM Na Caprylate | 94.3 ± 63.1 | 16 |
| +20 mM EDTA | 26.5 ± 20.1 | 4 |
| +20 mM Na Glycocholate | 21.6 ± 20.0 | 4 |
| +20 mM Lauric acid | 19.2 ± 10.2 | 3 |
| +20 mM Lysolecithin | 18.8 ± 22.0 | 3 |

| Sumatriptan (base) | | Enhancement Ratio |
|---|---|---|
| 6.8 mM | | _{Fs, base+enhancer/Fs, base alone} |
| Base alone | 1.6 ± 1.5 | |
| +20 mM Na Caprate | 2.0 ± 2.3 | 1.3 |
| +20 mM Lysolecithin | 1.8 ± 1.1 | 1.1 |
| +20 mM Na Glycocholate | 1.2 ± 1.0 | 0.8 |

• Values are presented as means ± SD with n ≧ 3.
• Steady state flux, Fₛ, was determined from the slope of the straight line (2-6 hrs) attained from the plot of the cumulative amount of drug permeated per unit area as a function of time.

In Table 1, F_{s, SS+enhancer} is the steady state flux value of the absorption of sumatriptan succinate when the donor-side contained sumatriptan succinate and an absorption enhancer. F_{s, SS alone} is the steady state flux value of the absorption of sumatriptan succinate when the donor-side contained sumatriptan succinate alone. F_{s, base+enhancer} is the steady state flux value of the absorption of sumatriptan base when the donor-side contained sumatriptan base and an absorption enhancer. F_{s, base alone} is the steady state flux value of the absorption of sumatriptan base when the donor-side contained sumatriptan base alone. The enhancement ratio is the ratio of the steady state absorption of the drug, e.g., sumatriptan succinate or sumatriptan base, when mixed with an absorption enhancer to the steady state absorption of the drug alone. As seen in Table 1, sodium caprate, sodium laurate, sodium caprylate, and sodium lauryl sulfate (SLS) enhanced the permeation of sumatriptan succinate across the buccal membrane. Sodium caprate showed the most dramatic effect in enhancing sumatriptan succinate absorption. EDTA, sodium glycocholate, lauric acid, and lysolecithin showed only minor enhancing effects on the permeation of sumatriptan succinate. However, the absorption of sumatriptan base was not significantly enhanced by the absorption enhancers.

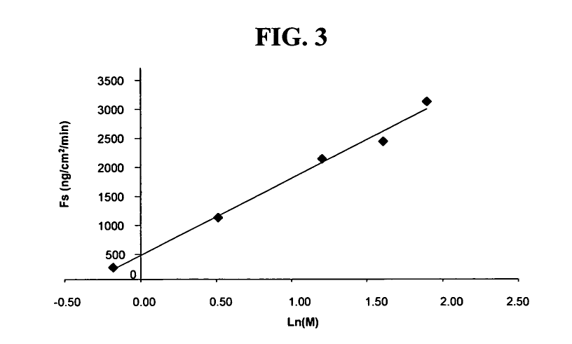

Table 2 summarizes the data from Table 1 for the experiment using sumatriptan succinate and sodium caprate, and provides values for molar ratio (M) and ln(M). The values for M were calculated from the experimental concentration of sumatriptan succinate used, i.e., 12 mM, and from the concentrations of sodium caprate used, i.e., 10 mM, 20 mM, 40 mM, 60 mM and 80 mM. The data in Table 2 is graphically depicted in FIG. 3, which provides a plot of Fₛ as a function of ln(M). In FIG. 3, the slope (the value for K) was 1329.2 ng/cm²/min; the intercept (the value for F₀) was 420 ng/cm²/min. Regression analysis showed that the correlation coefficient (r) was 0.9946.

TABLE 2
Steady state flux value (Fₛ) as a function of the molar
ratio (M) of a molar concentration of sodium caprate to
a molar concentration of sumatriptan succinate.

| Na Caprate | | | |
|---|---|---|---|
| (mM) | Molar Ratio (M) | In(M) | Fₛ (ng/cm²/min) |
| 10 | 0.83 | −0.18 | 210.4 |
| 20 | 1.67 | 0.51 | 1055.4 |
| 40 | 3.33 | 1.20 | 2083.4 |
| 60 | 5.00 | 1.61 | 2379.7 |
| 80 | 6.67 | 1.90 | 3068.6 |

Sumatriptan succinate = 12 mM

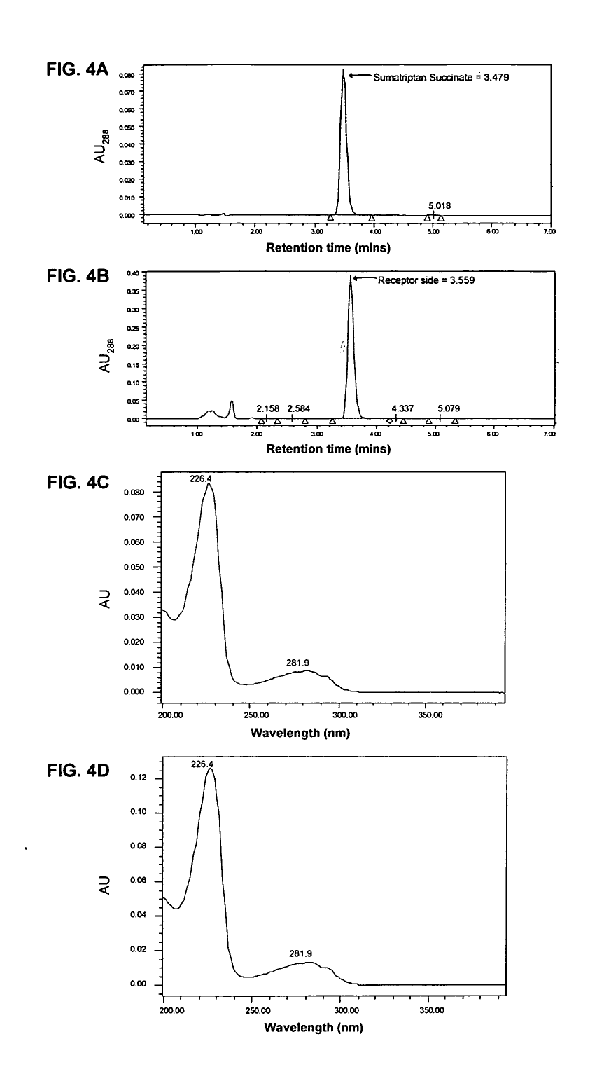

HPLC analysis was performed to compare the retention time for a standard solution of sumatriptan succinate alone (FIG. 4A) and the solution from the receptor-side of the side-by-side diffusion experiment (FIG. 4B). The retention time for sumatriptan succinate alone was 3.479 min and the retention time for the solution from the receptor-side was 3.559 min. FIGS. 4C and 4D provide the UV absorbance data (200-400 nm) for the major peaks observed in FIG. 4A and FIG. 4B, respectively. In both FIGS. 4C and 4D, two peaks corresponding to 226.4 nm and 281.9 nm were observed. The UV absorbance of the pure drug solution (FIG. 4C) was similar to the UV absorbance of the sample obtained from the receptor chamber (FIG. 4D).

### Example 2

### Mucoadhesive Formulations

Mucoadhesion allows buccal tablets to remain in close contact with the site of drug administration. Table 3 provides three mucoadhesive formulations, Rx1 to Rx3, for mucoadhesive, monolithic buccal tablets. Drug containing buccal tablets (patches) were placed on the buccal mucosa of the human volunteers, whereas, placebo buccal tablets (patches) were placed on the front upper gum of human volunteers to check residence time. Table 3 shows that the residence time of sumatriptan succinate on a buccal membrane can be varied by varying the type of mucoadhesives in the formulation. As seen in Table 3, HXF grade of hydroxypropyl cellulose (HPC) results in longer (60 min) residence time on the buccal mucosa compared to EXF grade of HPC, which exhibits lesser (30 min) residence time.

TABLE 3
Residence time of mucoadhesive formulations.

| | | Amount/Tablet (mg) | | |
|---|---|---|---|---|
| | Ingredients | R × 1 | R × 2 | R × 3 |
| 1 | Sumatriptan Succinate | 10 | 10 | 10 |
| 2 | Sodium Caprate | 10 | 10 | 10 |
| 3 | Polyethylene Glycol | 38.6 | 38.6 | 38.6 |
| | 8000 (PEG 8000) | | | |
| 4 | Anhydrous Lactose, | 15 | 15 | 15 |
| | NF (DT Grade) | | | |
| 5 | Poly(ethylene oxide) | 5 | 5 | 5 |
| | (POLYOX 301) | | | |
| 6 | Povidone K30 (PVP K30) | 15 | 5 | 5 |
| 7 | HPC (EXF) | | 10 | |
| 8 | HPC (HXF) | | | 10 |
| 9 | Talc | 2 | 2 | 2 |
| 11 | Aspartame | 3 | 3 | 3 |
| 12 | Flavor | 1.4 | 1.4 | 1.4 |

| | | | |
|---|---|---|---|
| Total Tablet Weight (mg) | 100 | 100 | 100 |
| Residence time on buccal membrane | 30 min | 30 min | 60 min |

A TA-XTplus Texture Analyzer (Texture Technologies Corporation, Scarsdale, N.Y.) was used to evaluate the effect of different pharmaceutical excipients on mucoadhesion. Table 4 provides the results of experiments using different pharmaceutical excipients on mucoadhesion of 5% ethylene oxide (POLYOX 301) (Union Carbide, Danbury, Conn.) in PEG 8000 based buccal tablets (formulations Rx4 through Rx13). The detachment force (g) is the force required to detach the tablet from a surface to which the tablet is attached, e.g., glass or mucosa. A buccal tablet was fixed onto the probe of a TA-XTplus Texture Analyzer and a 300 g force was applied for 2 min by pressing the tablet against a glass surface wetted by 0.1 ml purified water. The detachment force was determined by measuring the force required to detach the tablet from the glass surface.

The erosion time of the tablet matrix is a measure of the physical integrity of the drug dosage form. A standard USP dissolution apparatus type I (Paddle, 50 RPM) was used for determining the erosion time for complete erosion of the tablet in purified water (300 ml). The pharmaceutical dosage form was fixed onto the bottom of the dissolution vessel utilizing the wet adhesive property of the dosage form itself.

The results in Table 4 indicate that by varying the amount and types of mucoadhesive components in the tablet, the detachment force and erosion time of the tablet can be varied, e.g., povidone (PVP K30) and hydroxypropyl cellulose (HPC) have a synergistic effect on the mucoadhesion property of POLYOX 301.

TABLE 4
Detachment Force and Erosion Time of Buccal Tablets.

| | Ingredients | mg/Tablet | | | | | | | | | |
|---|---|---|---|---|---|---|---|---|---|---|---|
| No. | Formulation No. | R × 4 | R × 5 | R × 6 | R × 7 | R × 8 | R × 9 | R × 10 | R × 11 | R × 12 | R × 13 |
| 1 | Polyethylene oxide | 5 | 5 | 5 | 5 | 5 | 5 | 5 | 5 | 5 | 5 |
| | (POLYOX 301) | | | | | | | | | | |
| 2 | PEG 8000 | 95 | 47.5 | 47.5 | 47.5 | 47.5 | 47.5 | 47.5 | 47.5 | 32.0 | 47.5 |
| 3 | Maltodextrin (M100) | | 47.5 | 47.5 | | | | | | 32.0 | |
| 4 | Lactose DT | | | | 47.5 | | | | | | |
| 5 | Dextrose Anhydrous | | | | | 47.5 | | | | | |
| 6 | HPMC (3 cps) | | | | | | 47.5 | | | | |
| 7 | HPC (EXF) | | | | | | | 47.5 | | | |
| 8 | PVP K30 | | | | | | | | 47.5 | 32.0 | |
| 9 | Mannitol | | | | | | | | | | 47.5 |
| 11 | PRIMOJEL | | | 2 | | | | | | | |
| 12 | PRUV | 1 | 1 | 1 | 1 | 1 | 1 | 1 | 1 | 1 | 1 |
| | Tablet run weight (mg) | 100 | 100 | 100 | 100 | 100 | 100 | 100 | 100 | 100 | 100 |
| | Detachment Force (g) | 157 | 164 | 189 | 135 | 133 | 139 | 284 | 1031 | 480 | 170 |
| | Erosion Time (min) | 24 | 68 | 115 | 62 | 24 | 130 | 360 | 40 | 26 | 30 |

### Example 3

### Characterization of Formulations

Samples containing sumatriptan succinate alone, sodium caprate alone or compositions containing sumatriptan succinate and sodium caprate were analyzed by DSC, using a Universal V2.6D instrument from TA Instruments (Delaware, USA). In formulations prepared by the dry mixing process (FIG. 1), the location and area of the DSC thermogram peak corresponding to sumatriptan succinate were not significantly affected by the dry mixing process indicating that the crystallinity of sumatriptan succinate was not altered by the method of preparing the formulation. However, the DSC endotherm corresponding to sumatriptan succinate was not evident in samples prepared by the wet mixing process (FIG. 2), probably due to loss of crystallinity of the drug. Samples prepared by both the dry and wet methods showed enhancement or improvement in absorption of sumatriptan succinate as a function of the molar ratio of the molar concentration sodium caprate to the molar concentration of sumatriptan succinate.

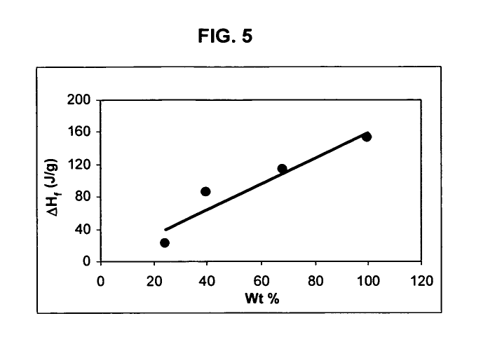

To determine the identity of peaks in the dry mixtures containing sumatriptan succinate and sodium caprate, the latent heat of fusion (ΔH_{f}) of the endotherm observed at about 170° C. in sumatriptan succinate alone and in the dry mixtures was examined as a function of the wt % of sumatriptan succinate. As seen in Table 5, the latent heat of fusion of peak at about 170° C. in the dry mixtures decreased as the wt % of sumatriptan succinate decreased, which indicates that the peak at about 170° C. corresponds to sumatriptan succinate. In Table 5, M is the molar ratio of the molar concentration of sodium caprate to the molar concentration of sumatriptan succinate. The data in Table 5 is graphically depicted in FIG. 5. In FIG. 5, a linear relationship is observed between ΔH_{f} of the dry mixtures and the wt % of sumatriptan succinate, which indicates that the peak at about 170° C. in the dry mixtures corresponds to sumatriptan succinate.

TABLE 5
ΔH_{f}-Wt % of sumatriptan succinate.

| | | | Wt % of | | |
|---|---|---|---|---|---|
| | | Molar | Sumatriptan | Peak | |
| | | Ratio (M) | Succinate | temp. (° C.) | ΔH_{f} (J/g) |
| 1 | Sumatriptan | | 100 | 170 | 153 |
| | succinate alone | | | | |
| | (SS) | | | | |
| 2 | Dry mixture | 1 | 68 | 167 | 113 |
| 3 | Dry mixture | 3.2 | 40 | 167 | 85 |
| 4 | Dry mixture | 6.6 | 24 | 169 | 22 |

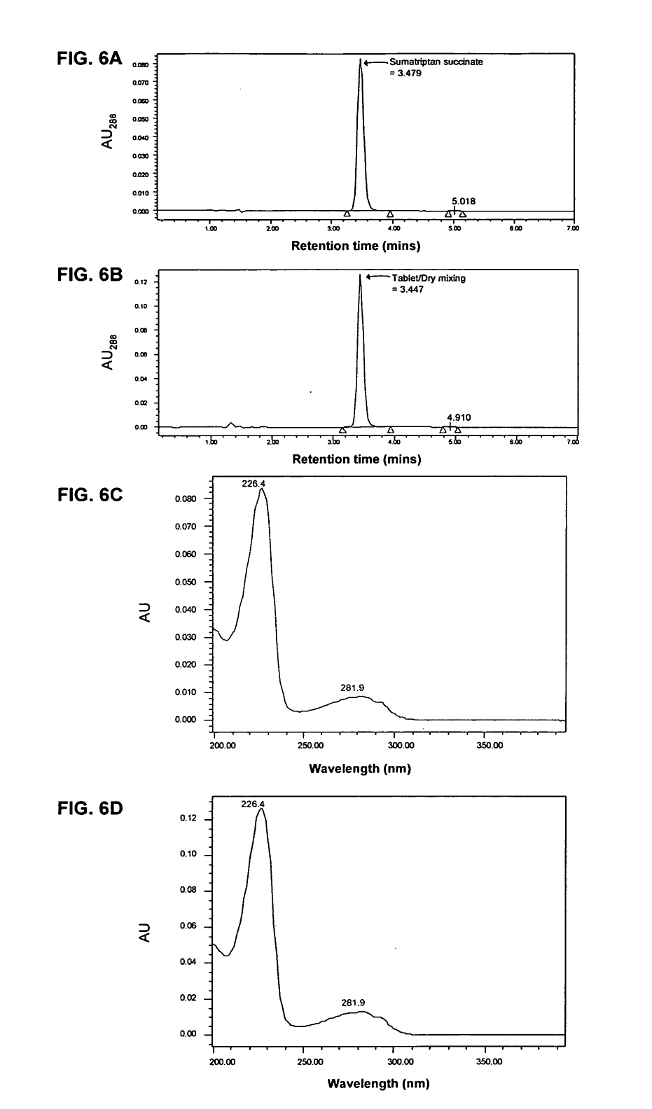

HPLC analysis was performed using a Waters 2695 separation module HPLC system equipped with a reverse phase C₁₈ column (150 mm×3.9 mm ID, 5 μm) (Waters Corp., Milford, Mass.). Ultraviolet analysis was performed using a Waters 2996 photodiode array detector (Waters Corp., Milford, Mass.) with the wavelength set at 228 nm to detect sumatriptan succinate. The retention time was determined for a standard solution of sumatriptan succinate alone (FIG. 6A) and for a dissolved sample of a dry mixture of sodium caprate and sumatriptan succinate prepared at a molar ratio (M) of 2.1 (FIG. 6B). The retention time was about 3.5 min at the HPLC condition stated above for both sumatriptan succinate alone and for the dry mix formulation. FIGS. 6C and 6D provide UV absorbance data (200-400 nm) for the major peak observed in FIG. 6A and FIG. 6B, respectively. In both FIGS. 6C and 6D, two peaks corresponding to 226.4 nm and 281.9 nm were observed. The UV absorbance of the solution containing drug alone (FIG. 6C) was similar to the UV absorbance of the formulation prepared by the dry mixing method (FIG. 6D).

A wet granulate of sumatriptan succinate and sodium caprate was prepared by dissolving sodium caprate and sumatriptan succinate in a molar ratio (M) of about 2.1 in 50% alcohol/50% H₂O, granulating with lactose, drying, milling, mixing with other excipients and compressing into a tablet.

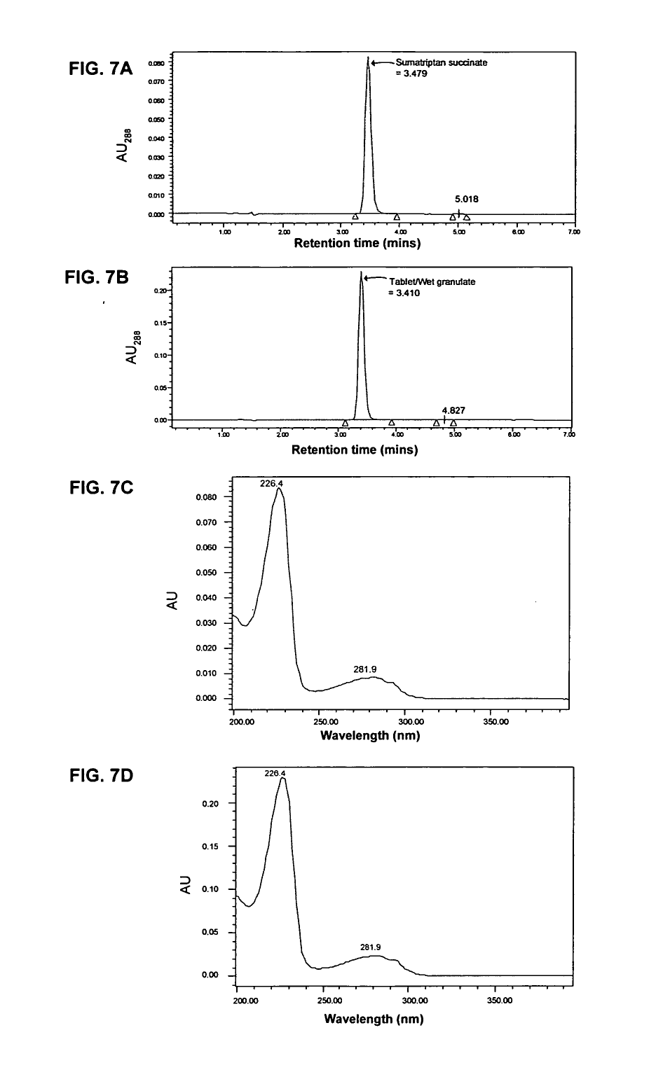

HPLC analysis was performed to compare the retention time for a standard solution of sumatriptan succinate alone (FIG. 7A) and a solution of the wet granulated product (FIG. 7B). The retention time for sumatriptan succinate alone was 3.479 min and the retention time for the wet mixture was 3.410 min. FIGS. 7C and 7D provide UV absorbance data (200-400 nm) for the major peak observed in FIG. 7A and FIG. 7B, respectively. In both FIGS. 7C and 7D, two peaks corresponding to 226.4 nm and 281.9 nm were observed. The UV absorbance of the solution containing drug alone (FIG. 7C) was similar to the UV absorbance of the wet granulated product (FIG. 7D).

### Example 4

### Sumatriptan Patent

Two in vivo dog studies were conducted. In the first study, the bioavailability of sumatriptan fast dissolving buccal tablet according to the present invention, and subcutaneous injection of sumatriptan (IMITREX, GlaxoSmithKline, United Kingdom, Brentford, Middlesex) were measured in conscious dogs (N=3). In the second study, the bioavailability of sumatriptan per-oral tablets (IMITREX, GlaxoSmithKline, United Kingdom, Brentford, Middlesex) and subcutaneous injection of sumatriptan (IMITREX, GlaxoSmithKline, United Kingdom, Brentford, Middlesex) were measured in anesthetized dogs (N=6). The dose of buccal and per-oral tablets was 25 mg per dog; the dose of subcutaneous injection was 6 mg per dog.

Male beagle dogs were used for all studies. The subcutaneous injection and per-oral tablets were commercial products under the trade name of IMITREX. Food was withheld from the dogs for a minimum of 12 hours before the study and during the study, and food was returned to them at 4 hours postdose. Water was supplied ad libitum.

Blood samples were collected from the dog's foreleg veing into heparin tubes; plasma samples were then separated at 4° C. centrifuge and kept at −60° C. to −80° C. until analysis. Sumatriptan was extracted from plasma via solid extraction and analyzed by LC/MS/MS. The pharmacokinetic parameters were determined using WinNonlin.

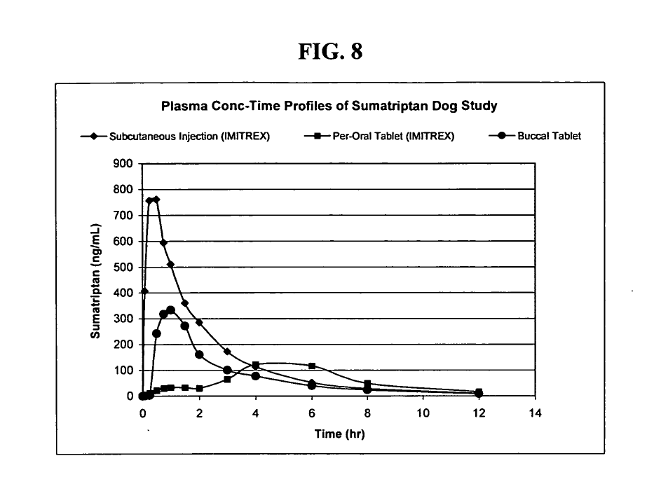

FIG. 8 shows the plasma concentration-time profiles of sumatriptan buccal tablets compared with subcutaneous and per-oral dosage forms. The buccal formulation used in the study was the fast dissolving buccal tablet of Table 6, which produced a residence time of 15 minutes in the dog buccal cavity. Table 7 shows the pharmacokinetic parameters of the evaluated sumatriptan dosage forms. In the dog studies, the fast dissolving buccal tablet displayed a significantly faster onset of Cₘₐₓ than the per-oral tablet; a Tₘₐₓ of 0.92 hr. was observed compared to 4.5 hrs in the per-oral tablets. In addition, the bioavailability of sumatriptan was increased 61% in the fast dissolving tablet compared to the per-oral tablet.

TABLE 6
Formulation Composition of Sumatriptan Succinate
Buccal Tablet Used in Dog Study

| SL # | Ingredients | mg/Tablet |
|---|---|---|
| 1 | Sumatriptan Succinate | 35 |
| 2 | Sodium Caprate | 15 |
| 3 | Polyethylene Glycol 8000 (PEG 8000) | 26 |
| 4 | Anhydrous Lactose, NF (DT Grade) | 15 |
| 5 | Poly(ethylene oxide) (Polyox 301) | 6.5 |
| 6 | Hydropropyl Cellulose (HPC) HXF | 19 |
| 7 | Talc | 4 |
| 8 | Aspartame | 3 |
| 9 | Strawberry Flavor | 1.5 |

| | |
|---|---|
| Total Tablet Weight (mg) | 125 |
| Residence time on dog buccal membrane | 15 min |

TABLE 7
Pharmacokinetic Parameters of Sumatriptan In Vivo Dog Study
(AUC values are dose-normalized)

| | | | | |
|---|---|---|---|---|
| | AUCₗₐₛₜ | | | |
| | (ng × | | | Ratio |
| Administration | hr/ml) | Cₘₐₓ (ng/ml) | Tₘₐₓ (hr) | AUC/AUC_{subcutaneous} |

Study 1 (N = 3)

| | | | | |
|---|---|---|---|---|
| IMITREX Subcutaneous | 845 ± 123 | 195 ± 53 | 0.25 | N/A |
| Injection - 6 mg | | | | |
| Buccal Tablet - 25 mg | 502 ± 177 | 456 ± 150 | 0.92 ± 0.52 | 0.61 ± 0.18 |

Study 2 (N = 6)

| | | | | |
|---|---|---|---|---|
| IMITREX Subcutaneous | 1007 ± 87 | 213 ± 57 | 0.50 ± 0.16 | N/A |
| Injection - 6 mg | | | | |
| IMITREX Per-Oral | 381 ± 112 | 183 ± 84 | 4.5 ± 1.2 | 0.38 ± 0.10 |
| Tablet - 25 mg | | | | |

While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A pharmaceutical composition for rapid transmucosal delivery comprising: sumatriptan succinate and sodium caprate,
wherein a molar ratio (M) of a molar concentration of the sodium caprate to a molar concentration of the sumatriptan succinate is about 0.1 or greater, wherein absorption of the sumatriptan succinate across a biological membrane (Fₛ) is equal to F₀+κln(M), wherein the F₀ is a steady state flux value of the absorption when the molar ratio of a molar concentration of sodium caprate to a molar concentration of sumatriptan succinate is 1, and wherein the κ is an enhancement factor.

2. The composition of claim 1, wherein the molar concentration of the sodium caprate is about 1 μM to about 250 mM.

3. The composition of claim 2, wherein the molar concentration of the sodium caprate is about 10 mM to about 80 mM.

4. The composition of claim 1, wherein the molar ratio is about 0.1 to about 15.

5. The composition of claim 4, wherein the molar ratio is about 0.5 to about 10.

6. The composition of claim 1, wherein the F₀ is about 100 ng/cm²/min to about 1000 ng/cm²/min.

7. The composition of claim 6, wherein the F₀ is about 150 ng/cm²/min to about 950 ng/cm²/min.

8. The composition of claim 7, wherein the F₀ is about 420 ng/cm²/min.

9. The composition of claim 1, wherein the κ is about 1000 ng/cm²/min to about 2000 ng/cm²/min.

10. The composition of claim 9, wherein the κ is about 1200 ng/cm²/min to about 1500 ng/cm²/min.

11. The composition of claim 10, wherein the κ is about 1300 ng/cm²/min.

12. The composition of claim 1, wherein a regression analysis of the absorption provides a correlation coefficient (r) of about 0.9 to about 1.

13. The composition of claim 12, wherein the correlation coefficient (r) is about 0.95 to about 1.0.

14. The composition of claim 1, wherein the biological membrane is an epithelial membrane.

15. The composition of claim 1, wherein the biological membrane is a buccal mucosal membrane.

16. The composition of claim 1, wherein the composition further comprises hydroxypropyl cellulose, hydroxyethyl cellulose, hydroxypropylmethyl cellulose, sodium carboxymethyl cellulose, methyl cellulose, polyvinylpyrrolidone, polyethylene glycol, vegetable oil, polyols, lactose or combinations thereof.

17. The composition of claim 1, which is a mucoadhesive.

18. The composition of claim 17, wherein the mucoadhesive comprises poly(ethylene oxide), polyvinylpyrrolidone, copovidone, carbomer, polycarbophil, hydroxypropyl cellulose, hydroxyethyl cellulose, sodium carboxymethyl cellulose, polyvinyl alcohol, or combinations thereof.

19. The composition of claim 1, further comprising a diluent.

20. The composition of claim 19, wherein the diluent comprises lactose, starch, polyethylene glycol, maltodextrin, dextrose, mannitol, xylitol, other polyols or combinations thereof.

21. The composition of claim 1, wherein the composition can be administered by a non-parenteral route.

22. The composition of claim 1, which is a tablet, disk, patch, film, wafer, gel, paste, or solution dosage form.

23. A pharmaceutical composition for rapid transmucosal delivery comprising: sumatriptan succinate and an absorption enhancer,
wherein a molar ratio (M) of a molar concentration of the absorption enhancer to a molar concentration of the sumatriptan succinate is about 0.1 or greater, wherein absorption of the sumatriptan succinate across a biological membrane (Fₛ) is equal to F₀+κln(M), wherein the F₀ is a steady state flux value of the absorption when the molar ratio of a molar concentration of sodium caprate to a molar concentration of sumatriptan succinate is 1, and wherein the κ is an enhancement factor.

24. The composition of claim 23, wherein the absorption enhancer comprises sodium caprate, sodium caprylate, sodium laurate, sodium lauryl sulfate or combinations thereof.

25. A method of making a pharmaceutical composition for rapid transmucosal delivery comprising sumatriptan succinate and sodium caprate, the method comprising:
mixing sumatriptan succinate and sodium caprate to form a mixture,
wherein a molar ratio (M) of a molar concentration of the sodium caprate to a molar concentration of the sumatriptan succinate is about 0.1 or greater, wherein absorption of the sumatriptan succinate across a biological membrane (Fₛ) is equal to F₀+κln(M), wherein the F₀ is a steady state flux value of the absorption when the molar ratio of a molar concentration of sodium caprate to a molar concentration of sumatriptan succinate is 1, and wherein the κ is an enhancement factor.

26. The method of claim 25, further comprising compressing the mixture into a pharmaceutical composition, wherein the mixture is a dry mixture.

27. The method of claim 25, further comprising compressing the mixture into a pharmaceutical composition, wherein the mixture is a wet granulate.

28. A pharmaceutical composition made by the method of claim 25.

29. The method of claim 25, wherein the mixture is a gel, a paste, or a solution.

30. A method of making a pharmaceutical composition for rapid transmucosal delivery comprising sumatriptan succinate and sodium caprate, the method comprising:
dispersing sumatriptan succinate and sodium caprate in water or a solvent to prepare a mixture; and
casting the mixture to form a pharmaceutical composition,
wherein a molar ratio (M) of a molar concentration of the sodium caprate to a molar concentration of the sumatriptan succinate is about 0.1 or greater, wherein absorption of the sumatriptan succinate across a biological membrane (Fₛ) is equal to F₀+κln(M), wherein the F₀ is a steady state flux value of the absorption when the molar ratio of a molar concentration of sodium caprate to a molar concentration of sumatriptan succinate is 1, and wherein the κ is an enhancement factor.

31. The method of claim 30, wherein the mixture is spray dried to form a second mixture.

32. The method of claim 31, further comprising compressing the second mixture into a pharmaceutical composition.

33. A pharmaceutical composition made by the method of claim 30.

34. A method of treating migraine, the method comprising administering the pharmaceutical composition of claim 1 to a person in need of the treatment.

35. A method of treating cluster headache episodes, the method comprising administering the pharmaceutical composition of claim 1 to a person in need of the treatment.

36. A pharmaceutical composition for rapid transmucosal delivery comprising sumatriptan succinate and sodium caprate, wherein the molar concentration of the sodium caprate is about 1 μM to about 250 mM.

37. A pharmaceutical composition for rapid transmucosal delivery comprising sumatriptan succinate and sodium caprate, wherein the amount of sodium caprate per dosage unit is about 1 μmol to about 250 mmol.
